(19) United States
(12) Patent Application Publication
(10) Pub. No.: US 2021/0052275 A1
(43) Pub. Date: Feb. 25, 2021

(54) CYSTOSCOPY NEEDLE HOLDER AND CLIP CUTTER

(71) Applicant: James R. Bollinger, Paoli, PA (US)

(72) Inventor: James R. Bollinger, Paoli, PA (US)

(21) Appl. No.: 16/545,035

(22) Filed: Aug. 20, 2019

(51) Int. Cl. A61B 17/128; A61B 1/307; A61B 17/04; A61B 18/20

(52) U.S. Cl. CPC A61B 17/1285; A61B 1/307; A61B 2017/00438; A61B 18/201; A61B 17/0469

(57) ABSTRACT

A cystoscopic needle holder and clip cutter is provided herein with jaws including a cutting tool and a grasping tool. The upper and lower jaws feature opposing proximal and distal surface portions that meet when the jaws close. The proximal surface portion of the upper jaw may include a blade, and the proximal surface portion of the lower jaw may include a groove that receives the blade. The distal surface portions of the upper and lower jaw each feature flat surfaces that meet together as a grasp. A shaft extending axially from a handle to the jaws houses a camera channel and laser fiber channel. A laser fiber may extend through an opening in the mouth of the jaw region to provide an additional cutting option. Surgical defects may be closed primarily with the needle holder, and migrated polymer ligation clips may be resected more efficiently with the clip cutting options.

### FIELD OF INVENTION

The present disclosure relates to a surgical device, and more particularly, to a cystoscopic needle holder and clip cutter configured for cutting polymer ligation clips and holding a suturing needle for surgical defect closure.

### BACKGROUND

In the area of cystoscopic surgical instruments, utilization of one instrument for use through the cystoscope that provides the combination of a needle holder and a polymer ligation clip cutter through the cystoscope is desirable, yet, in current practice, the combination of these functions is unavailable in one cystoscopic instrument. Accordingly, there is a need for a cystoscopic surgical instrument that holds the potential for performance of multiple procedures previously done with electrocautery/resection with or without laser fulguration, while also enabling the physician to facilitate removal of polymer ligation clips lodged inside the interior lining of the bladder wall.

### SUMMARY

There is provided according to the embodiments of the invention a cystoscopic needle holder and clip cutter for cutting polymer ligation clips and holding a suturing needle for surgical defect closure. The cystoscopic needle holder and clip cutter comprises an upper jaw and a lower jaw configured to opposably articulate. Each of the upper jaw and the lower jaw has a distal grasping surface and a proximal cutting surface. The cystoscopic needle holder and clip cutter further comprises a handle comprising a pair of finger loops operable to actuate a tensioning system to opposably articulate the upper jaw and the lower jaw. The cystoscopic needle holder and clip cutter further comprises a shaft having a proximal end and a distal end, where the shaft is connected to the handle at the proximal end of the shaft and connected to the upper jaw and the lower jaw at the distal end of the shaft. The cystoscopic needle holder and clip cutter further comprises a pair of channels disposed within the shaft that extend axially through the shaft, where each of the channels has an opening at the proximal end of shaft and an opening at the distal end of the shaft at an interior mouth region between the upper jaw and the lower jaw. The distal grasping surfaces are operable to hold a needle or polymer ligation clip when the upper jaw and the lower jaw opposably articulate. The proximal cutting surfaces are operable to cut a polymer ligation clip when the upper jaw and the lower jaw opposably articulate. Each of the channels is configured to slidably receive and hold a camera device or a laser fiber.

In an embodiment, the tensioning system comprises a ratchet mechanism disposed between the handle and the proximal end of the shaft.

In another embodiment, each of the proximal cutting surfaces of the upper jaw and the lower jaw further comprises a blade, wherein the blades of the upper jaw and the lower jaw are operable to provide a cutting force when the upper jaw and the lower jaw opposably articulate.

In another embodiment, the proximal cutting surface of the upper jaw further comprises a blade and the proximal cutting surface of the lower jaw further comprises a groove, wherein the groove receives the blade upon opposable articulation of the upper jaw and the lower jaw.

In yet another embodiment, the cystoscopic needle holder and clip cutter includes a jaw mechanism comprising the upper jaw and the lower jaw, wherein the jaw mechanism is deflectable.

Further, there is provided according to the embodiments of the invention a surgical device for use as part of a robotic cystoscopic instrument, further comprising an upper jaw and a lower jaw configured to opposably articulate. Each of the upper jaw and the lower jaw has a distal grasping surface and a proximal cutting surface. The surgical device further comprises a tensioning system operable to opposably articulate the upper jaw and the lower jaw. The surgical device further comprises a shaft having a proximal end and a distal end, where the shaft is connected to the upper jaw and the lower jaw at the distal end of the shaft. The surgical device further comprises a pair of channels disposed within the shaft that extend axially through the shaft, where each of the channels has an opening at the proximal end of shaft and an opening at the distal end of the shaft at an interior mouth region between the upper jaw and the lower jaw. The distal grasping surfaces are operable to hold a needle or polymer ligation clip when the upper jaw and the lower jaw opposably articulate. The proximal cutting surfaces are operable to cut a polymer ligation clip when the upper jaw and the lower jaw opposably articulate. Each of the channels is configured to slidably receive and hold a camera device or a laser fiber.

In an embodiment of the surgical device, the tensioning system comprises a ratchet mechanism disposed between the handle and the proximal end of the shaft.

In another embodiment of the surgical device, each of the proximal cutting surfaces of the upper jaw and the lower jaw further comprises a blade, wherein the blades of the upper jaw and the lower jaw are operable to provide a cutting force when the upper jaw and the lower jaw opposably articulate.

In another embodiment of the surgical device, the proximal cutting surface of the upper jaw further comprises a blade and the proximal cutting surface of the lower jaw further comprises a groove, wherein the groove receives the blade upon opposable articulation of the upper jaw and the lower jaw.

In yet another embodiment of the surgical device, the cystoscopic needle holder and clip cutter comprises a jaw mechanism comprising the upper jaw and the lower jaw, wherein the jaw mechanism is deflectable.

Also provided herein are methods for cutting and removing polymer ligation clips within the urinary bladder. A cystoscopic needle holder and clip cutter is provided, and a camera is inserted through a proximal cameral opening of a camera channel in a proximal end of a shaft of the cystoscopic needle holder and clip cutter. The cystoscopic needle holder and clip cutter is inserted into an accessory channel of a cystoscope, and a polymer ligation clip is visualized through the camera. The polymer ligation clip may then be cut with a proximal cutting surface of an upper jaw and a proximal cutting surface of a lower jaw of the cystoscopic needle holder and clip cutter, and a piece of a broken clip may then be grasped within the distal grasping surfaces of the upper jaw and lower jaw of the cystoscopic needle holder and clip cutter. The piece of the broken clip may then be resected by removing the cystoscopic needle holder and clip cutter from the bladder through the accessory channel of the cystoscope.

In another embodiment, the tensioning system of the cystoscopic needle holder and clip cutter comprises a ratchet mechanism disposed between the handle and the proximal end of the shaft. The method can include utilizing the handle to apply force from the ratchet mechanism through the jaws when cutting the clip with each of the proximal cutting surfaces of the upper jaw and the lower jaw of the cystoscopic needle holder and clip cutter. The method can further include grasping a piece of a broken clip with each of the distal grasping surfaces of the upper jaw and the lower jaw of the cystoscopic needle holder and clip cutter. The method can further include resecting the piece of the broken clip by removing the cystoscopic needle holder and clip cutter from the bladder through the accessory channel of the cystoscope.

In another embodiment, where each of the proximal cutting surfaces of the upper jaw and the lower jaw further comprises a blade, and the blades of the upper jaw and the lower jaw meet together to provide a cutting force upon opposable articulation of the upper jaw and the lower jaw, the method can include utilizing the blades of the upper jaw and lower jaw when performing a cutting maneuver during a polymer ligation clip resection.

In yet another embodiment, where the proximal cutting surface of the upper jaw further comprises a blade, and the proximal cutting surface of the lower jaw further comprises a groove, and the groove receives the blade to provide a cutting force upon opposable articulation of the upper jaw and the lower jaw, the method can include utilizing the blade of the of the upper jaw and groove of the lower jaw when performing a cutting maneuver during a polymer ligation clip resection.

In another embodiment, where a deflectable jaw mechanism of the cystoscopic needle holder and clip cutter comprises the upper jaw and the lower jaw, the method can further include deflecting the jaw mechanism of the cystoscopic needle holder and clip cutter to access and resect a polymer ligation clip when performing a cutting maneuver during a polymer ligation clip resection.

Also provided herein are methods for resecting a bladder tumor en bloc and closing a surgical defect primarily within the urinary bladder. A cystoscopic needle holder and clip cutter is provided, and a camera is inserted through a proximal cameral opening of a camera channel in a proximal end of a shaft of the cystoscopic needle holder and clip cutter. The cystoscopic needle holder and clip cutter is inserted into an accessory channel of a cystoscope, and the cystoscope is navigated with the camera to reach a bladder tumor. The cystoscopic needle holder and clip cutter is removed from the cystoscope through the accessory channel. A needle and suture is grasped with the cystoscopic needle holder and clip cutter, and the cystoscopic needle holder and clip cutter is reinserted into the accessory channel of the cystoscope while holding the needle and suture with the cystoscopic needle holder and clip cutter. The cystoscopic needle holder and clip cutter is utilized to make a stitch through the bladder tumor, and then the cystoscopic needle holder and clip cutter is removed from the cystoscope through the accessory channel of the cystoscope while grasping the needle and suture. The surgeon then manually pulls on the suture stitch attached to the tumor, and then the cystoscopic needle holder and clip cutter is reinserted into the accessory channel of the cystoscope. A determination is made whether to resect the tumor with the clip cutting option of the proximal cutting surfaces of the upper jaw and the lower jaw or the laser fiber option of the cystoscopic needle holder and clip cutter. The tumor is then resected with either the cutting option provided by the proximal cutting surfaces of the upper jaw and the lower jaw or the laser fiber option of the cystoscopic needle holder and clip cutter, and the cystoscopic needle holder and clip cutter is then removed from the cystoscope through the accessory channel of the cystoscope. The tumor is then removed from the bladder en bloc. The cystoscopic needle holder and clip cutter is then reinserted into the accessory channel of the cystoscope while holding the needle and suture with the cystoscopic needle holder and clip cutter, and the surgical defect is closed primarily with the cystoscopic needle holder and clip cutter.

In another embodiment, the tensioning system of the cystoscopic needle holder and clip cutter comprises a ratchet mechanism disposed between the handle and the proximal end of the shaft. The method can include utilizing the handle to apply force from the ratchet mechanism through the jaws when grasping the needle and suture with the cystoscopic needle holder and clip cutter and when closing the surgical defect primarily with the cystoscopic needle holder and clip cutter.

In another embodiment, where each of the proximal cutting surfaces of the upper jaw and the lower jaw further comprises a blade, and the blades of the upper jaw and the lower jaw meet together to provide a cutting mechanism upon opposable articulation of the upper jaw and the lower jaw, the method can include utilizing the blades of the of the upper jaw and the lower jaw when performing a cutting maneuver during a bladder tumor resection.

In yet another embodiment, where wherein the proximal cutting surface of the upper jaw further comprises a blade, and the proximal cutting surface of the lower jaw further comprises a groove, and the groove receives the blade to provide a cutting force upon opposable articulation of the upper jaw and the lower jaw, the method can include utilizing the blade of the upper jaw and the groove of the lower jaw when performing a cutting maneuver during a bladder tumor resection.

In another embodiment, where a deflectable jaw mechanism of the cystoscopic needle holder and clip cutter comprises the upper jaw and the lower jaw, the method can further include deflecting the jaw mechanism of the cystoscopic needle holder and clip cutter to access and resect a bladder tumor when performing a cutting maneuver during a bladder tumor resection.

These and other objects, features and advantages of the present invention will be apparent from the following detailed description of illustrative embodiments thereof, which is to be read in connection with the accompanying drawings.

### BRIEF DESCRIPTION OF THE DRAWINGS

The present invention is illustrated by way of example, and not by way of limitation, in the figures of the accompanying drawings in which:

FIG. 1A is a perspective view of an embodiment of the cystoscopic needle holder and clip cutter, where the handle with finger rings joins the shaft in a parallel orientation with respect to the opened jaws, such that an imaginary plane extending through both of the finger rings also extends through both of the jaws when the jaws are in the open position.

FIG. 1B is a perspective view of an embodiment of the cystoscopic needle holder and clip cutter, where the handle with finger rings joins the shaft in a perpendicular orientation with respect to the jaws, such that an imaginary plane extending through both of the finger rings is perpendicular to the plane extending through both of the jaws when the jaws are in an open position.

FIG. 2 is an expanded view of an embodiment of the jaws of the cystoscopic needle holder and clip cutter, where the jaws are in an open position.

FIG. 3 is an expanded view of an embodiment of the jaws of the cystoscopic needle holder and clip cutter, where the jaws are in an open position and the distal portion of the jaws may be configured to hold a needle and the proximal portion of the jaws may be configured to cut a polymer ligation clip.

FIG. 4 is an additional expanded view of an embodiment of the jaws of the cystoscopic needle holder and clip cutter, where the jaws are in an open position and the distal portion of the jaws may be configured to hold a needle and the proximal portion of the jaws may be configured to cut a polymer ligation clip.

FIG. 5 is an expanded view of an embodiment of the jaws of the cystoscopic needle holder and clip cutter, where the jaws are in a closed position.

FIG. 6 is an expanded view of an embodiment of the jaws of the cystoscopic needle holder and clip cutter, providing an enface perspective view of the interior of the mouth of the jaws, where the jaws are in open position.

FIG. 7 is a perspective view of an embodiment of the needle holder and clip cutter with a laser fiber extending distally through the laser fiber channel within the shaft and beyond the jaws to a target location.

FIG. 8 is a perspective view of an embodiment of the needle holder and clip cutter, where the jaw mechanism is deflectable.

FIG. 9 is a flow diagram of an example procedure to remove a polymer ligation clip, which is used in conjunction with the disclosed embodiments.

FIG. 10 is a flow diagram of an example procedure to perform a transurethral resection of a bladder tumor and primary closure, which is used in conjunction with the disclosed embodiments.

### DETAILED DESCRIPTION

Various embodiments of the invention are described in the following paragraphs. Where like elements have been depicted in multiple embodiments, identical or similar reference numerals have been used for ease of understanding.

In the present era of robotic surgery, the instrumentation for cystoscopic procedures has lagged behind in terms of what could be done through the urethra. In the last twenty years, the field of urologic surgery has expanded to include robotic applications, so that many procedures previously done through an incision are only done infrequently that way. Now, robotic surgery typically requires only several small incisions into which small surgical tools and a camera may be inserted, so that no large incision is required. In particular, laparoscopic procedures have seen substantial progress in the area of robotic surgery.

Cystoscopic procedures, however, are done much the same way they were done twenty years ago. This is partially due to the fact that laparoscopic procedures involve approximately six surgical openings, for six different surgical tools, whereas cystoscopy procures generally only have one opening through the urethra, to accompany one instrument at a time. Cystoscopic procedures are thus limited by the single point of entry for the surgical tools. Accordingly, it would be advantageous to have a cystoscopy instrument, for both manual and robotic surgical applications, that features a needle holder, polymer ligation clip cutter and laser fiber in one instrument, so that one instrument could be inserted to achieve multi-purpose functionality. Notably, the cystoscopic needle holder and clip cutter of the embodiments described herein may provide new surgical functionality for cystoscopic procedures, as there is currently no such cystoscopic instrument for transurethral applications. While endoscopic instruments may generally be used for laparoscopic procedures through the abdominal wall, or for ear, nose and throat procedures, the cystoscopic needle holder and clip cutter of the embodiments described herein may be specifically designed with the appropriate dimensions, such as the length of the shaft and diameter of the channels within the shaft, to accommodate new functionality and potential for transurethral cystoscopic procedures. In comparison, laparoscopic instruments generally implement a longer shaft with a larger diameter. The parameters and functionality of a cystoscopic instrument are different from those of a lap aroscopic instrument.

Accordingly, the development of a needle holder that can be used through the cystoscope holds the potential for performance of multiple procedures previously done with electrocautery/resection with or without laser fulguration. A cystoscopic needle holder would allow suturing through the cystoscope, to do for example, a ureteral advancement type of re-implantation, excision of full thickness bladder wall, so that pathology can obtain the whole tumor for histology without contaminating the whole pelvis with tumor cells and other likely applications. More specifically, the development of a cystoscopic needle holder with cutting option may enable the physician to perform a transurethral en bloc resection of bladder tumor and primary closure.

The potential for a transurethral en bloc resection of bladder tumor and primary closure with application of the cystoscopic needle holder with cutting option of the embodiments disclosed herein may enable the pathologists to “stage” the bladder tumor if they receive the whole tumor as it is in the patient, as opposed to a handful of tissue pieces. The ability of the pathologists to receive the whole tumor will allow them to see exactly where the tumor penetrates into the muscle layer of the bladder. The pathologists will thus not lose such valuable information obtained by studying the whole tumor because of distortion of the sample by the resection method.

Further, a transurethral en bloc resection of bladder tumor and primary closure with application of the cystoscopic needle holder offers the potential for reducing bladder tumor recurrences. In general, one of the issues with bladder tumors is recurrences, 30% of the time, at least. In attempt to identify the cause of a recurrence, urologists determine if the recurrent tumor is in the same site as the first tumor. One reason noted by urologists for the propensity for recurrence is the condition of the transitional cell lining of the bladder being altered at some current or previous time by exposure to chemicals in cigarette smoke, the prime example, even if the patient no longer smokes. However, an additional reason may be due to the manner in which the first tumor was initially resected, particularly where the tumor was removed in pieces, thereby contaminating the whole pelvis with tumor cells.

To address the issue of contamination of the pelvis and bladder lining when tumors are resected in pieces, en bloc resection of bladder tumor and primary closure with application of the cystoscopic needle holder may expand upon laparoscopic techniques. For example, in robotic surgery urologists place a tissue specimen in a plastic bag, known as a LapSac, which is passed into the abdomen on a shaft thru the laparoscopic port and opened inside the patient, so that the resected tissue can be placed inside the LapSac, and it will not be exposed to the wound channel during removal of the tissue. Applying the same principle to bladder tumors, the propensity of recurrence in bladder tumor resection in the traditional manner, where the tumor is removed in pieces, is due to contamination of the normal bladder mucosa by tumor cells to which the normal bladder mucosa is exposed in a traditional transurethral resection of bladder tumor procedure. Accordingly, removal of the specimen thru the cystoscope in one piece, thus not exposing the naked tumor cells to the rest of the bladder lining holds potential to reduce bladder tumors recurrences, so that cystoscopic procedures may be performed more like laparoscopic procedures, so that they may be more beneficial for patients.

In addition, the ability to close surgical defects by suturing in the cystoscopic context holds additional potential for improved patient outcomes. Currently, with the traditional cystoscopic approach, the surgical site, such as a resection point where unwanted tissue is removed, heals secondarily. More specifically, the wound heals slowly over a period of weeks with an increase in the risk of bleeding and infection. In particular, current cystoscopic surgical procedures, such as bladder tumor resections, rely on electrocautery to cauterize the surgical defect. However, the electrosurgical application cannot close the wound, and leaves it open to heal with a scar. The development of a cystoscopic needle holder would enable the urologist to close the surgical defect via suturing rather than electrocautery. Defect closure may be accomplished in less time with fewer complications by suturing, while electrocautery requires healing over the course of several weeks (e.g., 6-8 weeks).

Moreover, the ability to close a surgical defect by suturing becomes significant when the patient is taking anti-coagulants for a cardiac condition that is secondary to the primary urologic issue. For example, a patient might be anti-coagulated (i.e., taking a blood thinner) because the patient had a stroke or has an arrhythmia. An arrhythmia causes blood to stagnate in the auricular appendage, so the irregular heart may potentially send a blood clot to the brain and lead to a stroke. Accordingly, because the anti-coagulation issue is one the urologists encounter, the ability to use a cystoscopic needle holder and suture will reduce bleeding, cause less pain and lower the risk of infection in anti-coagulated patients. In contrast, when a bladder tumor is electro-resected and the wound is cauterized in an anti-coagulated patient, there is more risk of bleeding, as there is a raw tissue surface that is open to the urine, and the tissue has to heal secondarily over the course of several (e.g., 6-8) weeks. When the surgical defect can be closed via suturing, the wound can heal within a week (e.g., 3-5 days).

Further, a cystoscopic needle holder and suture would enable the patient to avoid extended time with the post-operative catheter, which is used to drain the bladder following surgery, to drain blood and prevent clotting and clot retention, so that the bladder does not stretch while the wound is healing. By closing the surgical defect with the needle holder and suture, the wound will heal faster, thereby enabling the bladder wall to hold urine normally without relief from the post-operative catheter.

In addition, a cystoscopic needle holder and suture would enable the patient to avoid complications due to the obturator nerve reflex. More specifically, when removing a tumor from the bladder, a wire loop that is about ⅜ of an inch in diameter is typically used to remove the tumor via electro-resection. By introducing a cystoscopic needle holder that would enable the surgeon to close the surgical defect primarily following resection of the tumor, the surgeon would no longer require electrocautery to close the wound. For example, when cauterizing a wound with electrocautery with the aforementioned wire loop, there is an electrical reflex, as the electrical energy is delivered to the lining of the bladder or prostate at a precise location, but the electrical current which diffuses beyond the bladder is taken away by a grounding plate. In the context of the obturator reflex, there is a nerve just beyond the wall of the bladder called the obturator nerve that runs to the muscles of the thigh. When the current hits the nerve, it activates the nerve and causes the muscles to contract. Such muscle contraction pushes the wall of the bladder into the resecting loop while the patient is asleep. The surgeon must be very careful of this scenario when removing a tumor on side wall of bladder, as the obturator reflex can cause the bladder to contract onto the loop, making a hole in the bladder wall and potentially damaging the obturator vessels on the outside of the bladder.

Thus, a cystoscopic needle holder would provide urologists significant functional advantages. For example, ureteral advancement for reflux, excision of bladder diverticuli and tumors, resection of full thickness bladder wall tissue for biopsy, and resection are a few procedures that could be done endoscopically closing the surgical defect primarily. Utilizing absorbable barbed and looped sutures with a pop off option for the needle can facilitate these procedures and most likely avoid the post-operative catheter. Further, the absence of the need for electrocautery would eliminate the obturator nerve reflex, which causes the bladder to contract over the electrical resection loop causing a bladder perforation.

An additional need in the field of urologic surgery concerns the cutting and removal of polymer ligation clips, such as Weck hemostatic clips, that have eroded and migrated into the bladder. More specifically, urologic surgeons use the polymer ligation clips laparoscopically outside of the bladder on the vessels during a prostatectomy. The clips may be applied manually or robotically to clip the prostatic vascular pellicles. In particular, the polymer clip has one end with a jaw that engages the opposing end of the clip, so that the clip locks. Upon removing the prostate, due to a tumor for example, the surgeon typically uses a ligation clip on the vessels outside of the bladder to stop the bleeding. Because the clip is plastic and inert, the surgeon may leave it inside the patient indefinitely, as it is not considered a foreign body that will be attacked and rejected by the patient's immune system. Right now, urologists have no instrument to efficiently remove such clips. Removal of a clip is time consuming and cumbersome for the physician.

Although the clip is secured to the vessels and is initially outside the bladder, it may still migrate through the bladder wall over the course of several years because it erodes into the lumen of the bladder, as it work its way through the wall of the bladder and into the interior, sometimes causing hematuria and obstruction of the urethra. Further, once the clip has migrated through the bladder wall, the clip then becomes encrusted, as solutes in the urine that have the ability to form a stone attach to the clip and form a stone. The stone may then cause blood in the urine, infection and urgency and frequency. Although less than 10% of prostatectomies result in clip migration through the bladder wall, numerous prostatectomies are performed around the world, robotically and manually, so the incidence of clip migration through the bladder wall is an ongoing concern.

As a hypothetical example, a patient may have a prostatectomy, and then five years later, the patient will present with the aforementioned symptoms: blood in the urine, infection and urgency and frequency. First, a urologist will find hematuria (i.e., blood in the urine), so the urologist will send the patient to a radiologist for a CT scan. Upon examining the patient with a CT scan, the radiologist will typically not discover the migrated clip, as the radiologist generally looks for a large stone or tumor instead. Moreover, because the clips are plastic, they do not have the same density and reflective characteristics for purposes of the CT scan. Therefore, based on the patient's history of having a prostatectomy, the radiologist expects the plastic clip to be located outside the bladder where it was initially placed during the prostatectomy, but the radiologist cannot clearly see on the CT scan that the clip has migrated through the lining of the bladder wall. Next, the radiologist will recommend an endoscopic examination or cystoscopy. Subsequently, the urologist will examine the patient with a cystoscope and find a migrated clip, which requires removal from the inner lining of the bladder wall through the cystoscope. Removal of the clip from the tissue of the interior bladder wall may leave a minor lesion, but generally no suturing is required.

Currently, to remove the migrated and encrusted clip from the inner lining of the bladder tissue, the clip must be broken at the angle portion of the clip. In particular, there are two ends or tips to the clip, which typically do not erode. In contrast, the angle portion is usually the portion that erodes and becomes encrusted with a stone. Current cystoscopic forceps are not designed to bear the level of force required to break the polymer ligation clip so that it may be removed. In particular, the clip is too large to be removed through the cystoscope without first being broken into at least two smaller pieces. Therefore, when the urologist finds a migrated clip with a stone on the angle portion, the urologist will use a laser to remove the stone from the angle portion. Once the stone has been removed, the clip, itself, can be cut at the angle portion with the laser after approximately fifteen minutes with application of the laser. Once the clip is broken, the surgeon can attempt to grasp the individual pieces with a cystoscopic biopsy forceps. However, such forceps available now are not designed for this purpose and may not be strong enough to cut the clip and pull the pieces from the bladder tissue. The forceps available now are not designed for this maneuver.

Accordingly, urologic surgeons require an improved cystoscopic instrument to facilitate removal of the polymer ligation clips from the bladder, as there is currently no designated instrument for this procedure. Further, urologists require an instrument that reduces the amount of time required to break the encrusted clip into separate pieces. As aforementioned, cutting the encrusted clip with a laser requires approximately fifteen minutes. It would be beneficial to both the patient and surgeon to simplify and reduce the amount of time required to cut and remove a migrated, encrusted clip from the inner lining of the bladder.

To provide further functionality for the cystoscopic needle holder and clip cutter, an embodiment of the needle holder with polymer hemostatic clip cutter may also include a laser fiber extending axially along the length of the shaft and through an opening in the needle holder to provide optional functionality. The laser fiber may be a 200 Holmium laser fiber. For example, the instrument may enable the physician to lase stone encrustation from a polymer hemostatic clip in conjunction with cutting and/or breaking the clip with the clip cutter. In addition, the laser fiber extending through an opening in the needle holder may enable the physician to perform resections and electrocautery, in conjunction with closing a surgical defect primarily with the needle holder. The energy provided by the aforementioned laser fiber would not stimulate the obturator nerve reflex. To further compliment the functionality of the laser fiber, the cystoscopic needle holder and clip cutter of the proposed embodiments may also feature a thirty degree camera lens, which may be navigated through a channel extending axially along the length of the shaft through an additional opening in the needle holder.

Accordingly, urologic surgeons require a cystoscopic needle holder and clip cutter that may be passed through a cystoscope to provide the operator the ability to close surgical defects primarily with suturing and to transect polymer hemostatic clips that have migrated into the bladder neck. In particular, the jaws of the instrument may be constructed so that the proximal portion of the jaw has a sharp edge to cut polymer clips. The distal portion of the jaw may have a textured surface to allow a barbed looped suture with pop-off needle to be passed through the cystoscope, so that the suture can be used to close resection sites. Following defect closure, the textured surface of the distal portion of the jaw may then be used to grasp and remove the needle through the cystoscope.

In addition, the handles of the instrument may operate in conjunction with a ratcheted closure mechanism to allow for holding of tissue. In particular, the instrument may feature a reinforced ratchet mechanism to support jaws that may grasp the polymer clip with the distal end of the jaws to pull the clip loose and a sharp edge at the proximal portion of the jaws that may be used to cut the polymer clip. Further, because such an instrument may be used cystopically to pass through the urethra, rather than the abdominal wall, the instrument may be smaller in diameter and length than a typical lap aroscopic instrument. The jaw mechanism of the instrument may also be deflectable, for enhanced navigation and ergonomic functionality within the bladder.

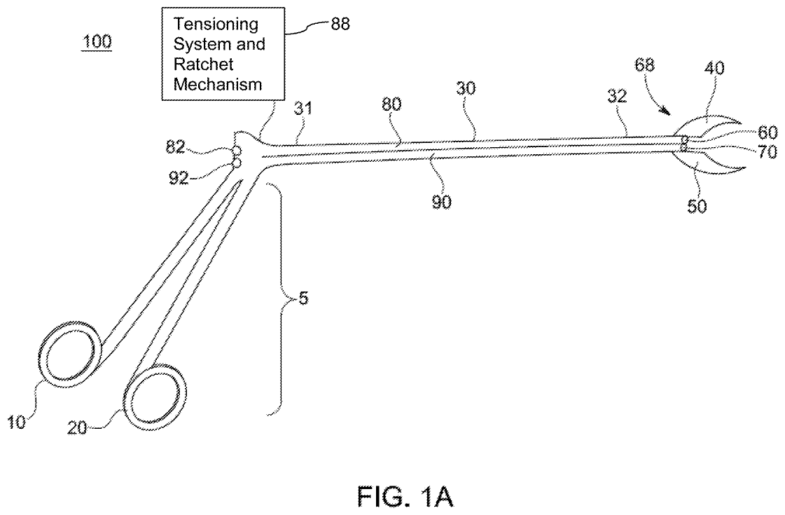

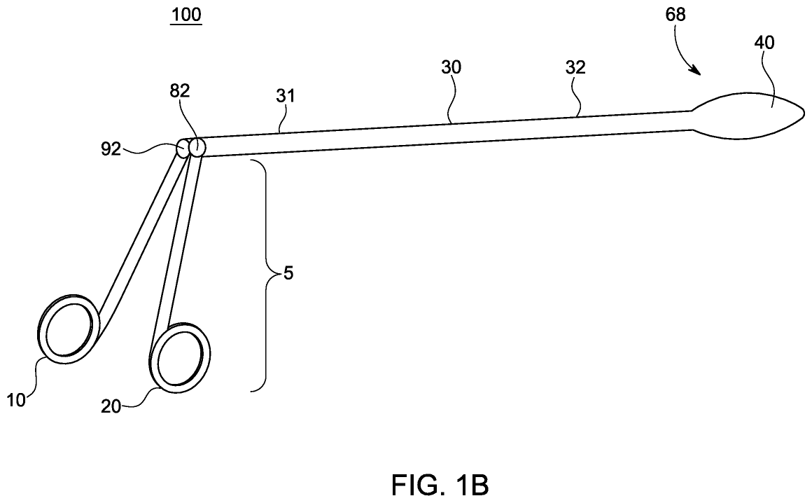

Referring to FIG. 1A, a perspective view of a cystoscopic needle holder and clip cutter 100 configured for cutting polymer ligation clips and holding a suturing needle for surgical defect closure is shown. The instrument 100 features finger rings 10, 20 of a handle 5 that joins a shaft 30. The shaft 30 extends axially and distally to an upper jaw 40 and a lower jaw 50. The upper and lower jaws 40, 50 function by moving in opposition to each other and may be used for grasping and cutting. It should be understood that the upper and lower jaws 40, 50 may each be any shape, such as a convex shape as shown in FIG. 1A, for example.

In the embodiment of FIG. 1A, the handle 5 with finger rings 10, 20 is parallel to the jaws 40, 50, such that an imaginary plane extending through both of the finger rings 10, 20 also extends through both of the jaws 40, 50 when the jaws 40, 50 are in the open position. In FIG. 1B, the handle 5 with finger rings 10, 20 may join the shaft 30 in a position such that the handle 5 with finger rings 10, 20 is perpendicular to the jaws 40, 50, so that the imaginary plane extending through both of the finger rings 10, 20 is perpendicular to the plane extending through both of the jaws 40, 50 when the jaws are in an open position. As shown in FIG. 1B, the perpendicular orientation of the handle 5 and finger rings 10, 20 with respect to the jaws 40, 50 may provide alternative ergonomic orientation for the positioning of the surgeon's hand and fingers in relation to the action of the jaws 40, 50. The length of the shaft 30 is a standard, known length, relative to and appropriate for the cystoscopic application of the instrument 100. In addition, the diameter of the shaft 30 may accommodate a 24 French (i.e., 8 millimeter diameter) cystoscope.

Referring again to FIG. 1A, the instrument 100 may be comprised of a metal alloy, such as titanium or galvanized stainless steel, for example. Because the physician controls and manipulates the handle 5 with finger rings 10, 20 to perform surgical maneuvers with the jaws 40, 50 of the instrument 100, a reinforced tensioning system, such as a ratchet mechanism 88 may be disposed between the handle 5 and the proximal end 31 of the shaft 30 to provide dexterity, control and strength of movement for the actions of the jaws 40, 50. The reinforced ratcheted mechanism 88 may facilitate holding of tissue and grasping and cutting of a polymer ligation clip, for example. In particular, the reinforced ratcheted mechanism 88 may facilitate firmly grasping a polymer ligation clip within the distal portion of the jaws 40, 50 to pull the polymer ligation clip loose from the tissue. The reinforced ratcheted mechanism 88 may also facilitate firmly grasping a needle within the distal portion of the jaws 40, 50 for use in closing surgical defects primarily. The reinforced ratcheted mechanism 88 may also allow the finger rings 10, 20 of the handle 5 to lock when the jaws 40, 50 are holding a polymer ligation clip or needle. In addition, the reinforced ratcheted mechanism 88 may also facilitate cutting of a polymer ligation clip within the sharp, cutting edges of the proximal portion of the jaws 40, 50. Known mechanisms for operation of the jaws 40, 50 to achieve the desired angle of separation between the jaws and the proper force include pivots as well as levers or pantographs. Such known mechanisms accomplish operation of the jaws 40, 50 based upon two opposite and combined actions. Those of skill in the art of surgical biopsy forceps instruments would understand that the jaws 40, 50 may alternate between an open and closed position relative to the corresponding pressure applied by the surgeon to the finger rings 10, 20 of the handle 5. For example, as the physician's thumb and index finger squeeze the finger rings 10, 20, the ratchet mechanism 88 transfers the force applied by the physician on the finger rings 10, 20 to the jaws 40, 50 through a pin or wire within the shaft 30. The pin or wire couples to the jaws 40, 50 and conveys the force applied by the physician on the finger rings 10, 20 of the handle 5 to the jaws 40, 50 to grasp tissue, hold a needle to close surgical defects primarily, or cut and resect polymer ligation clips.

Referring again to FIG. 1A, the shaft 30 may also include a camera channel 80 for inserting a camera with thirty-degree lens into a proximal camera opening 82 on the shaft 30 that extends to a distal camera opening 60. The camera channel 80 may slidably receive the camera through the proximal camera opening 82. The shaft 30 may also include a laser fiber channel 90 for inserting a laser fiber into a proximal laser fiber opening 92 on the shaft 30 that extends to a distal laser fiber opening 70. The laser fiber channel 90 may slidably receive the laser fiber through the proximal laser fiber opening 92. Each of the camera channel 80 and laser fiber channel 90 may be approximately three millimeters in diameter. In particular, the camera channel 80 may be at least three millimeters in diameter to accommodate a three-millimeter camera lens. The laser fiber may be a 200 Holmium laser fiber, for example. The laser fiber channel 90 may be approximately three millimeters in diameter to accommodate a high-power laser fiber, for situations where the physician may determine that a high power laser fiber is required to lase larger stone encrustation on a polymer ligation clip, for example. A higher power laser fiber is larger than the aforementioned 200 Holmium laser fiber. Accordingly, the laser fiber channel 90 may accommodate the appropriate laser fiber, as selected by the physician. The laser fiber may be advanced axially and distally along the length of the shaft 30 through the distal laser fiber opening 70 and beyond the jaws 40, 50 to a target location on the tissue, such as a bladder tumor, a surgical defect for closure by electrocautery or a polymer ligation clip. The additional functionality of the laser fiber may enable the physician to lase stone encrustation from a polymer ligation clip and perform resections and electrocautery, in conjunction with closing a surgical defect primarily with the needle holder. The target location and maneuvers of the laser fiber, needle holder and clip cutter may be visualized by the thirty-degree lens on the screen of the camera tower in the operating room. Further, power is supplied to the laser fiber from a laser generator in the operating room, and a technician sets the laser frequency and power setting on the laser generator. The camera and lens within the camera channel 82 are powered by a camera tower in the operating room that provides a fiber optic light source for the camera, along with a screen for visualization of images captured by the camera. Both the camera tower and laser generator are standard equipment. In the embodiment shown in FIG. 1A, the camera channel 80 is superior (i.e., above) the laser fiber channel 90. In other embodiments, however, the laser fiber channel may be positioned above the camera channel. Alternatively, in another embodiment, the laser channel and camera channel may be positioned in a side-by-side orientation.

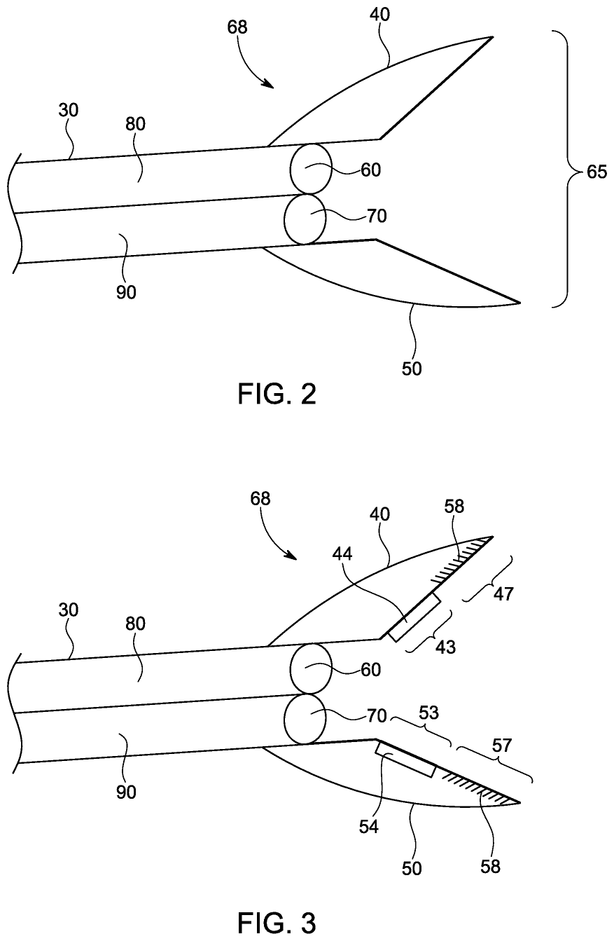

FIG. 2 provides an expanded view of the jaws 40, 50 of the cystoscopic needle holder and clip cutter 100, where upper jaw 40 and lower jaw 50 are in an open position, so that the interior mouth region 65 is visible. The jaws 40, 50 may each be approximately one centimeter in length. Further, the jaws 40, 50 may each feature a pointed distal tip as shown in FIG. 2. In other embodiments, the distal tips of the jaws 40, 50 may be rounded or curved. Within the interior mouth region 65 are the distal thirty-degree camera lens opening 60 and distal laser fiber opening 70. The physician may view the action of the upper and lower jaws 40, 50 and laser fiber 73 (shown in FIG. 7) through the distal thirty-degree camera opening 60 provided for the thirty-degree camera lens.

Referring to FIG. 3 showing an additional expanded view of the jaws 40, 50, the upper and lower jaws 40, 50 may also feature a cutting tool and a grasping tool. In particular, the upper jaw 40 features a proximal surface portion 43 and a distal surface portion 47 on the interior of the upper jaw 40 surface, and the lower jaw 50 features a proximal surface portion 53 and a distal surface portion 57 on the interior of the lower jaw 50 surface. The proximal surface portion 43 of the upper jaw 40 may include a sharp ridge or blade 44, and the proximal surface portion 53 of the lower jaw 50 may include a groove 54 that may be configured to receive the blade 44 of the opposing proximal surface portion 43 of the upper jaw 40, so that when the upper and lower jaws 40, 50 close together, the blade 44 of the upper jaw 40 is received within the groove 54 of the lower jaw 50. When the physician articulates the upper and lower jaws 40, 50 so that the blade 44 is received within the groove 54, the physician can apply a cutting force to an object, such as a polymer ligation clip, surgical suture or tissue, for example.

In addition, FIG. 3. also portrays the distal surface portion 47 of the upper jaw 40 and the distal surface portion 57 of the lower jaw 50. The distal surface portion 47 of the upper jaw 40 and the distal surface portion 57 of the lower jaw 50 may each feature a flat surface, so that when the upper and lower jaws 40, 50 close together, the flat surfaces of the distal surface portion 47 of the upper jaw 40 and the distal surface portion 57 of the lower jaw 50 meet together. In an embodiment the surfaces of the distal surface portion 47 of the upper jaw 40 and the distal surface portion 57 of the lower jaw 50 may include a denticulate texture 58 for enhanced traction and grasping functionality for holding a needle, gripping tissue or grasping of polymer ligation clips. Of course, the denticulate texture 58 as shown in FIG. 3 is exemplary, and various textures may be implemented to facilitate the grasping functionality of the instrument 100.

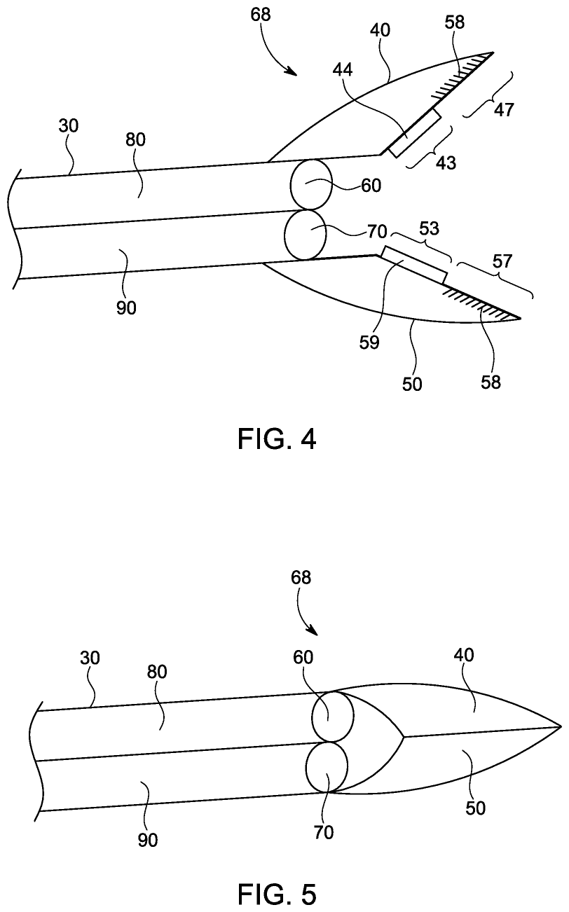

Referring to FIG. 4, an embodiment of the cystoscopic needle holder and clip cutter 100 is shown, where the upper and lower jaws 40, 50 include a cutting tool and a grasping tool. In this embodiment, the upper jaw 40 also features a proximal surface portion 43 and a distal surface portion 47 on the interior of the upper jaw 40 surface, and the lower jaw also 50 features a proximal surface portion 53 and a distal surface portion 57 on the interior lower jaw 50 surface. The proximal surface portion 43 of the upper jaw 40 may include a sharp ridge or blade 44, and the proximal surface portion 53 of the lower jaw 50 may similarly include an opposing blade 59 that may be configured to meet the blade 44 of the opposing proximal surface portion 43 of the upper jaw 40, so that when the upper and lower jaws 40, 50 close together, the blades 44, 59 provide enhanced functionality to cut objects, such as polymer ligation clips and sutures, for example. When the physician articulates the upper and lower jaws 40, 50 so that the blades 44, 59 meet, the physician can apply a cutting force to a polymer ligation clip, surgical suture or tissue.

FIG. 5 provides an expanded view of the jaws 40, 50 of the cystoscopic needle holder and clip cutter 100, where upper jaw 40 and lower jaw 50 are shown in a closed position. When the jaws 40, 50 are in the closed position, the distal thirty-degree camera opening 60 and distal laser fiber opening 70 are positioned behind the jaws 40, 50 within the shaft 30. The physician may view the action of the upper and lower jaws 40, 50 and laser fiber through the distal thirty-degree camera opening 60 that is provided for the thirty-degree lens.

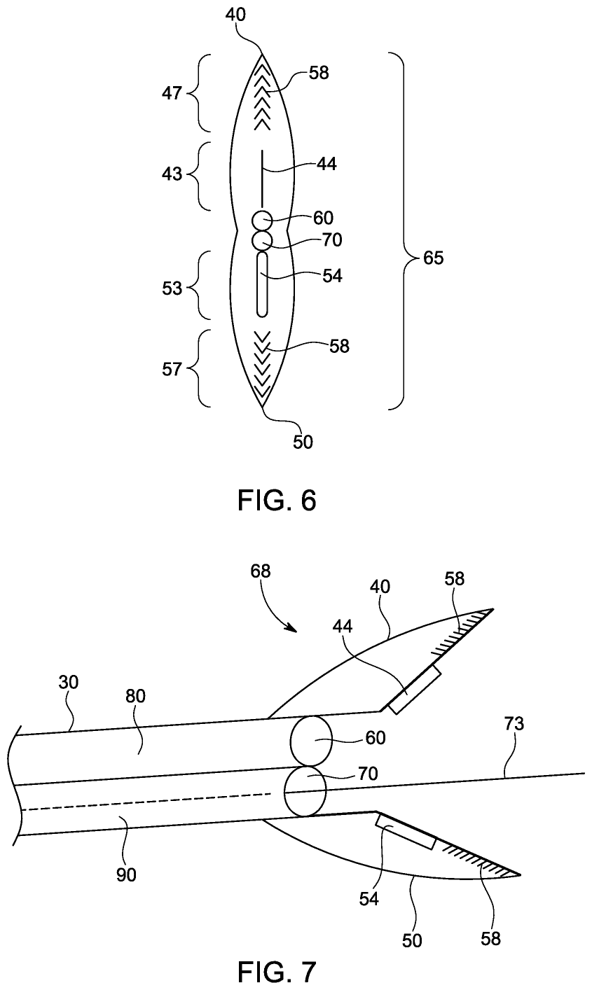

FIG. 6 provides an enface perspective view of an embodiment of the needle holder and clip cutter 100, where the upper jaw 40 and lower jaw 50 are portrayed in an open position, so that the interior mouth region 65 is visible. Within the interior mouth region 65 are the distal thirty-degree camera opening 60 and distal laser fiber opening 70. The physician may view the action of the upper and lower jaws 40, 50 and laser fiber through the distal thirty-degree camera opening 60 that is provided for the thirty-degree camera lens.

Referring again to FIG. 6, the upper jaw 40 may feature a proximal surface portion 43 and a distal surface portion 47 on the interior of the upper jaw 40 surface, and the lower jaw 50 may feature a proximal surface portion 53 and a distal surface portion 57 on the interior lower jaw 50 surface. The proximal surface portion 43 of the upper jaw 40 may include a sharp ridge or blade 44, and the proximal surface portion 53 of the lower jaw 50 may include a groove 54 that may be configured to receive the blade 44 of the opposing proximal surface portion 43 of the upper jaw 40, so that when the upper and lower jaws 40, 50 close together, the blade 44 of the upper jaw 40 is received within the groove 54 of the lower jaw 50. Of course and as shown in FIG. 4, the proximal surface portion 53 of the lower jaw 50 may similarly include an opposing blade 59 that may be configured to meet the blade 44 of the opposing proximal surface portion 43 of the upper jaw 40, so that when the upper and lower jaws 40, 50 close together, the blades 44, 59 provide enhanced functionality to cut polymer ligation clips and sutures, for example.

Referring once again to FIG. 6, the distal surface portion 47 of the upper jaw 40 and the distal surface portion 57 of the lower jaw 50 may each feature a flat surface, so that when the upper and lower jaws 40, 50 close together, the flat surfaces of the distal surface portion 47 of the upper jaw 40 and the distal surface portion 57 of the lower jaw 50 meet together. In an embodiment, the surfaces of the distal surface portion 47 of the upper jaw 40 and the distal surface portion 57 of the lower jaw 50 may have a denticulate texture 58 for enhanced traction and grasping functionality for holding a needle, gripping tissue or grasping of polymer ligation clips.

FIG. 7 provides a perspective view of an embodiment of the needle holder and clip cutter 100 with a laser fiber 73 extending through the proximal laser fiber opening 92 and through the distal laser fiber opening 70. The laser fiber 73 may be advanced axially and distally through the laser fiber channel 90 along the length of the shaft 30 and beyond the jaws 40, 50 to a target location on the tissue, such as a bladder tumor, a surgical defect for closure by electrocautery or a polymer ligation clip. It is understood that the target location and maneuvers of the laser fiber 73, and jaws 40, 50 of the needle holder and clip cutter 100 may be visualized by the thirty-degree lens on a screen of a camera tower. As aforementioned, power is supplied to the laser fiber 73 from a laser generator in the operating room, and a technician sets the laser frequency and power setting on the laser generator. In the embodiment shown in FIG. 7, the camera channel 80 is superior (i.e., above) the laser fiber channel 90. In other embodiments, however, the laser fiber channel may be positioned above the camera channel. Alternatively, in another embodiment, the laser channel and camera channel may be positioned in a side-by-side orientation.

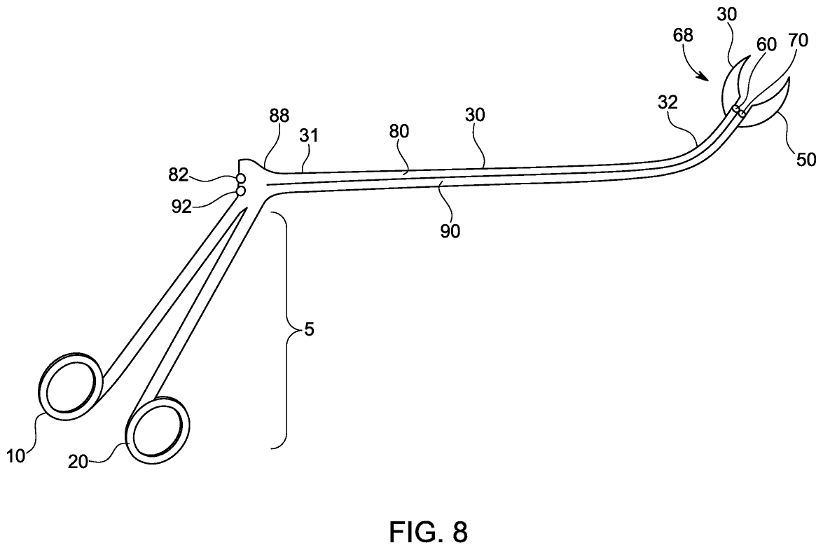

FIG. 8 is a perspective view of an embodiment of the needle holder and clip cutter, where the jaw mechanism is deflectable. Those of skill in the art would recognize that the jaw mechanism 68 of the instrument 100, including the jaws 40, 50, may also be deflectable without bending of the cystoscope, for enhanced navigation and ergonomic functionality within the bladder. The ability to maneuver the jaw mechanism 68 of the instrument 100 off the axis of the cystoscope may provide the physician enhanced tangential access to remove polymer ligation clips and/or perform resections and electrocautery, in conjunction with closing a surgical defect primarily with the needle holder.

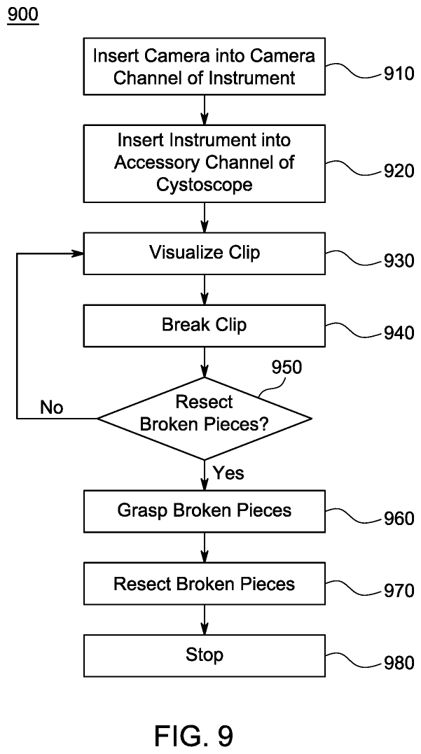

Referring to FIG. 9, a flow diagram illustrates the process 1000 of maneuvering the jaws of the instrument 100 to grasp, cut and remove a polymer ligation clip. As shown in FIG. 9, the process 900 begins 910 by inserting a camera with thirty-degree lens into the camera channel 80 of the instrument 100 by inserting the camera into a proximal camera opening 82 on the shaft 30 that extends to a distal camera opening 60. Next, the instrument 100 with camera placed inside the camera channel 80 is then inserted into an accessory channel of a cystoscope 920. The physician navigates the cystoscope by visualizing the navigation through the camera with thirty-degree lens within the camera channel 80, to navigate the beak of the cystoscope to reach a polymer ligation clip target within the bladder. After inserting 920 the instrument into the cystoscope, the physician then utilizes the camera to visualize the clip 930. After the instrument 100 has been inserted 910, the camera has been placed 920, and the clip can be visualized 930, the clip will be broken 940 with either or both of the clip cutter at the proximal portions 43, 53 of the jaws 40, 50 and the laser fiber 73. In particular, if the physician identifies the presence of stone encrustation on the clip, the physician may decide to use the laser fiber 73 to lase the stone encrustation from the clip before utilizing the clip cutter at the proximal portions 43, 53 of the jaws 40, 50 to break the clip. Next, the physician breaks the clip 940, either with the clip cutter, alone, or in conjunction with operation of the laser fiber 73. After the clip has been broken 940, a determination 950 will be made whether any additional pieces of the broken clip need to be broken further, so that the pieces are small enough to be resected through the cystoscope. Finally, the physician maneuvers the jaws 40, 50 to grasp 960 the pieces of the clip before resecting 970 the pieces out of the bladder and through the cystoscope.

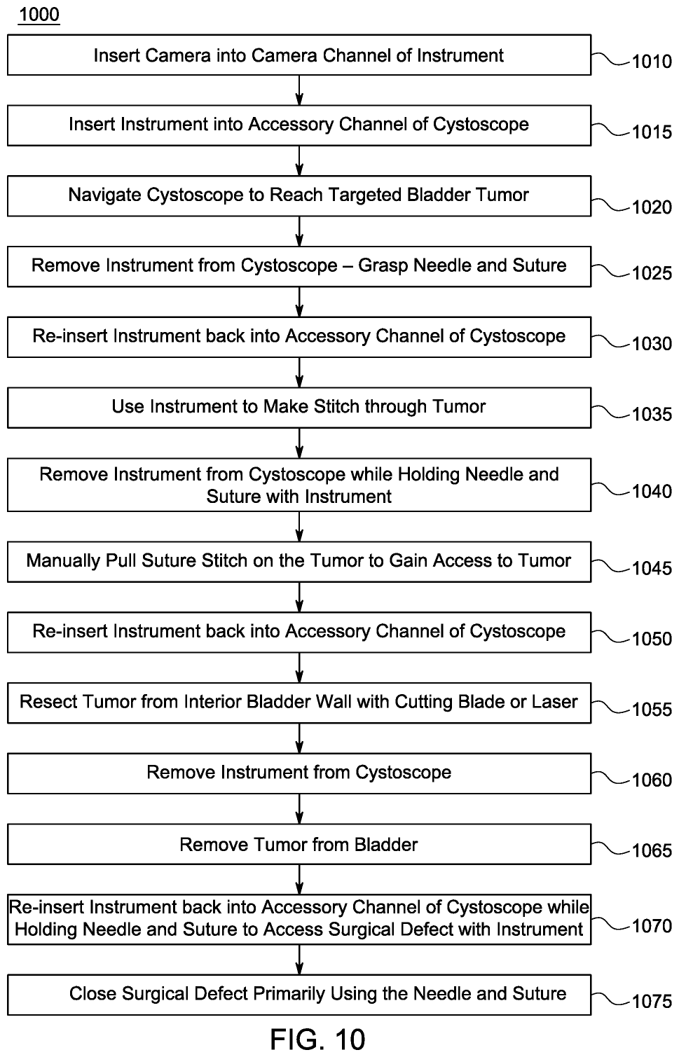

Referring to FIG. 10, a flow diagram illustrates the process 1000 of performing a transurethral resection of bladder tumor and primary closure. The process 1000 described in FIG. 10 may also be applied when performing a transurethral en bloc resection of bladder tumor. As shown in FIG. 10, the process 1000 begins 1010 by inserting a camera with thirty-degree lens into the camera channel 80 of the instrument 100 by inserting the camera into a proximal camera opening 82 on the shaft 30 that extends to a distal camera opening 60. Next, the instrument 100 with camera placed inside the camera channel 80 is then inserted into an accessory channel of a cystoscope 1015. The physician navigates the cystoscope by visualizing the navigation through the camera with thirty-degree lens within the camera channel 80, to navigate the beak of the cystoscope to reach a targeted bladder tumor, within the bladder interior 1020. After navigating the cystoscope with the camera to reach the bladder tumor 1020, the physician then removes the instrument 100 through the cystoscope so that the needle holder of the instrument 100 can be utilized to grasp a needle and suture 1025. Next, the physician reinserts 1030 the instrument 100 back into the previously placed cystoscope, which is now positioned at a target region for a tumor resection within the bladder. Then, the physician utilizes the needle holder of the instrument 100 to make a stitch 1035 through the tumor and then pulls the instrument 100 back out of the cystoscope while holding the needle and suture 1040. In particular, at this stage of the procedure, there are now two ends of the suture outside of cystoscope, as a stitch was made with the suture in the tumor, thereby effectively looping the suture through the tumor and back out of the cystoscope. Next, the physician manually pulls the suture that is stitched through the tumor 1045, so that the tumor is pulled into the interior (i.e., the middle) of the bladder, for better access to the tumor. The physician then reinserts the instrument 100 back into the cystoscope 1050. Once the instrument 100 is placed within the cystoscope at the target region for tumor resection, a determination will be made whether to resect the tumor with the proximal surface portions 43, 53 of the upper and lower jaws 40, 50 that may include the blade 44 as a cutting option, as shown in FIGS. 3 and 4, or to resect the tumor using the cutting option provided by the laser fiber 73, as shown in FIG. 7. Once the tumor has been resected 1055, the instrument 100 may be taken back out 1060 of the cystoscope. The resected tumor may then be removed from the bladder 1065 through the cystoscope. Finally, the physician may then reinsert the instrument 100 back into the cystoscope while holding a needle and suture to access the surgical defect 1070. The physician then closes the surgical defect primarily using the needle and suture 1075.

Urologic surgeons may also require the aforementioned cystoscopic needle holder and clip cutter 100 for use during robotic transurethral surgery. Using the ideas of a single port abdominal surgery platform, transurethral surgery can be developed into an enhanced robotics platform by utilizing improved instruments, such as the cystoscopic needle holder and clip cutter 100 of the proposed embodiments. In another embodiment, the cystoscopic needle holder and clip cutter 100 with cutting options provided by the proximal cutting surfaces 43, 53 of the upper and lower jaws 40, 50 and the laser fiber 73 for cutting polymer hemostatic clips, surgical sutures and tissue may be configured for robotic transurethral surgery for resections and electrocautery, in conjunction with closing a surgical defect primarily with the needle holder. Such transurethral robotic applications are disclosed in U.S. Pat. No. 9,956,042, which is incorporated by reference herein. In an embodiment of the cystoscopic needle holder 100 that is adapted for robotic transurethral surgery, it is understood that the cystoscopic needle holder 100 may not include the handle 5 with finger rings 10, 20, as a robotic mechanism may actuate a reinforced tensioning system, such as a ratchet mechanism 88 to provide dexterity, control and strength of movement for the actions of the jaws 40, 50.

Having thus described the presently preferred embodiments in detail, it is to be appreciated and will be apparent to those skilled in the art that many physical changes, only a few of which are exemplified in the detailed description of the invention, could be made without altering the inventive concepts and principles embodied therein. It is also to be appreciated that numerous embodiments incorporating only part of the preferred embodiment are possible which do not alter, with respect to those parts, the inventive concepts and principles embodied therein. The present embodiments and optional configurations are therefore to be considered in all respects as exemplary and/or illustrative and not restrictive, of the scope of the invention being indicated by the appended claims rather than by the foregoing description, and all alternate embodiments and changes to this embodiment which come within the meaning and range of equivalency of said claims are therefore to be embraced therein.

Although features and elements are described above in particular combinations, each feature or element can be used alone without the other features and elements or in various combinations with or without other features and elements.

1. A cystoscopic needle holder and clip cutter comprising:
an upper jaw and a lower jaw configured to opposably articulate and each of the upper jaw and the lower jaw having a distal grasping surface and a proximal cutting surface further comprising a sharp ridge or blade;
a shaft having a diameter configured for insertion within a cystoscope suitable for transurethral insertion, the shaft further comprising a proximal end and a distal end, the distal end connected to the upper jaw and the lower jaw at the distal end of the shaft;
a handle connected to the proximal end of the shaft including a pair of finger loops operable to actuate a tensioning system comprising a ratchet mechanism disposed between the handle and the proximal end of the shaft to
a pair of channels disposed within the shaft and extending axially through the shaft, each of the channels having an opening at the proximal end of the shaft and an opening at the distal end of the shaft at an interior mouth region between the upper jaw and the lower jaw,
wherein the distal grasping surfaces are operable to hold a needle and suture or a polymer ligation clip when the upper jaw and the lower jaw opposably articulate;
wherein the sharp ridges or blades of the upper jaw and the lower jaw provide a cutting force to cut a polymer ligation clip when the upper jaw and the lower jaw opposably articulate; and
wherein each of the channels is configured to slidably receive and hold one of a camera device or a laser fiber.

2. (canceled)

3. (canceled)

4. The cystoscopic needle holder and clip cutter of claim 1, wherein the proximal cutting surface of the upper jaw further comprises a sharp ridge or blade and the proximal cutting surface of the lower jaw further comprises a groove, wherein the groove receives the sharp ridge or blade upon opposable articulation of the upper jaw and the lower jaw.

5. The cystoscopic needle holder and clip cutter of claim 1, further comprising a jaw mechanism comprising the upper jaw and the lower jaw, wherein the jaw mechanism is deflectable.

6. A surgical device for use as part of a robotic cystoscopic instrument, further comprising:
an upper jaw and a lower jaw configured to opposably articulate and each of the upper jaw and the lower jaw having a distal grasping surface and a proximal cutting surface further comprising a sharp ridge or blade;
a shaft having a diameter configured for insertion within a cystoscope suitable for transurethral insertion, the shaft further comprising a proximal end and a distal end, the distal end connected to the upper jaw and the lower jaw at the distal end of the shaft;
a tensioning system comprising a ratchet mechanism disposed at the proximal end of the shaft operable to opposably articulate the upper jaw and the lower jaw; and
a pair of channels disposed within the shaft and extending axially through the shaft, each of the channels having an opening at the proximal end of the shaft and an opening at the distal end of the shaft at an interior mouth region between the upper jaw and the lower jaw,
wherein the distal grasping surfaces are operable to hold a needle and suture or a polymer ligation clip when the upper jaw and the lower jaw opposably articulate;
wherein the sharp ridges or blades of the upper jaw and the lower jaw provide a cutting force to cut a polymer ligation clip when the upper jaw and the lower jaw opposably articulate; and
wherein each of the channels is configured to slidably receive and hold one of a camera device or a laser fiber.

7. (canceled)

8. (canceled)

9. The surgical device of claim 6, wherein the proximal cutting surface of the upper jaw further comprises a blade and the proximal cutting surface of the lower jaw further comprises a groove, wherein the groove receives the blade upon opposable articulation of the upper jaw and the lower jaw.

10. The surgical device of claim 6, further comprising a jaw mechanism comprising the upper jaw and the lower jaw, wherein the jaw mechanism is deflectable.

11. A method for cutting and removing a polymer ligation clip within a urinary bladder, the method comprising:
providing a cystoscopic needle holder and clip cutter according to claim 1;
inserting the camera device through a proximal opening of a camera channel within the shaft of the cystoscopic needle holder and clip cutter;
inserting the cystoscopic needle holder and clip cutter into an accessory channel of a cystoscope;
visualizing the polymer ligation clip through the camera device;
cutting the polymer ligation clip with the proximal cutting surface of each of the upper jaw and the lower jaw of the cystoscopic needle holder and clip cutter;
grasping a piece of a broken clip with the distal grasping surface of each of the upper jaw and the lower jaw of the cystoscopic needle holder and clip cutter; and
resecting the piece of the broken clip by removing the cystoscopic needle holder and clip cutter from the bladder through the accessory channel of the cystoscope.

12. The method of claim 11, wherein the tensioning system of the cystoscopic needle holder and clip cutter comprises a ratchet mechanism disposed between the handle and the proximal end of the shaft, further comprising:
utilizing the handle to apply a force from the ratchet mechanism through the jaws;
cutting the polymer ligation clip with the proximal cutting surface of each of the upper jaw and the lower jaw of the cystoscopic needle holder and clip cutter;
grasping the piece of the broken clip with the distal grasping surface of each of the upper jaw and the lower jaw of the cystoscopic needle holder and clip cutter; and
resecting the piece of the broken clip by removing the cystoscopic needle holder and clip cutter from the bladder through the accessory channel of the cystoscope.

13. The method of claim 11, wherein the proximal cutting surface of each of the upper jaw and the lower jaw further comprises a blade and each of the blades of the upper jaw and the lower jaw meets together to provide a cutting force upon opposable articulation of the upper jaw and the lower jaw, further comprising:
utilizing the blades of the upper jaw and the lower jaw when performing a cutting maneuver during a polymer ligation clip resection.

14. The method of claim 11, wherein the proximal cutting surface of the upper jaw further comprises a blade and the proximal cutting surface of the lower jaw further comprises a groove and the groove receives the blade upon opposable articulation of the upper jaw and the lower jaw, further comprising:
utilizing the blade of the upper jaw and the groove of the lower jaw when performing a cutting maneuver during a polymer ligation clip resection.

15. The method of claim 11, wherein a deflectable jaw mechanism of the cystoscopic needle holder and clip cutter comprises the upper jaw and the lower jaw, further comprising:
deflecting the jaw mechanism of the cystoscopic needle holder and clip cutter to access and resect the polymer ligation clip when performing a maneuver during a polymer ligation clip resection.

16. A method for resecting a bladder tumor en bloc and closing a surgical defect primarily within a urinary bladder, the method comprising:
providing a cystoscopic needle holder and clip cutter according to claim 1;
inserting the camera device through a proximal opening of a camera channel within the shaft of the cystoscopic needle holder and clip cutter;
inserting the cystoscopic needle holder and clip cutter into an accessory channel of a cystoscope;
navigating the cystoscope with the camera device to reach a bladder tumor;
removing the cystoscopic needle holder and clip cutter from the cystoscope through the accessory channel;
grasping the needle and suture with the cystoscopic needle holder and clip cutter;
reinserting the cystoscopic needle holder and clip cutter into the accessory channel of the cystoscope while holding the needle and suture with the cystoscopic needle holder and clip cutter;
utilizing the cystoscopic needle holder and clip cutter to make a suture stitch through the bladder tumor;
removing the cystoscopic needle holder and clip cutter from the cystoscope through the accessory channel of the cystoscope while grasping the needle and suture;
pulling on the suture stitch attached to the bladder tumor;
reinserting the cystoscopic needle holder and clip cutter into the accessory channel of the cystoscope;
determining whether to resect the bladder tumor with a cutting option of the proximal cutting surface of each of the upper jaw and the lower jaw or a laser fiber option of the cystoscopic needle holder and clip cutter;
resecting the bladder tumor with either the cutting option or the laser fiber option of the cystoscopic needle holder and clip cutter;
removing the cystoscopic needle holder and clip cutter from the cystoscope through the accessory channel of the cystoscope;
removing the bladder tumor from the bladder en bloc;
reinserting the cystoscopic needle holder and clip cutter into the accessory channel of the cystoscope while holding the needle and suture with the cystoscopic needle holder and clip cutter; and
closing the surgical defect primarily with the cystoscopic needle holder and clip cutter.

17. The method of claim 16, wherein the tensioning system of the cystoscopic needle holder and clip cutter comprises a ratchet mechanism disposed between the handle and the proximal end of the shaft, further comprising:
utilizing the handle to apply a force from the ratchet mechanism through the jaws;
grasping the needle and suture with the cystoscopic needle holder and clip cutter; and
closing the surgical defect primarily with the cystoscopic needle holder and clip cutter.

18. The method of claim 16, wherein the proximal cutting surface of each of the upper jaw and the lower jaw further comprises a blade and each of the blades of the upper jaw and the lower jaw meets together to provide a cutting force upon opposable articulation of the upper jaw and the lower jaw, further comprising:
utilizing the blades of the upper jaw and the lower jaw when performing a cutting maneuver during a bladder tumor resection.

19. The method of claim 16, wherein the proximal cutting surface of the upper jaw further comprises a blade and the proximal cutting surface of the lower jaw further comprises a groove and the groove receives the blade upon opposable articulation of the upper jaw and the lower jaw; further comprising:
utilizing the blade of the upper jaw and the groove of the lower jaw when performing a cutting maneuver during a bladder tumor resection.

20. The method of claim 16, wherein a deflectable jaw mechanism of the cystoscopic needle holder and clip cutter comprises the upper jaw and the lower jaw, further comprising:
deflecting the jaw mechanism of the cystoscopic needle holder and clip cutter to access and resect the bladder tumor when performing a maneuver during a bladder tumor resection.

21. The shaft of claim 1 having a diameter configured for transurethral insertion through a Fr. 24 cystoscope.

22. The shaft of claim 6 having a diameter configured for transurethral insertion through a Fr. 24 cystoscope.

23. The pair of channels disposed within the shaft of claim 1 each having a diameter of approximately 3 millimeters.

24. The pair of channels disposed within the shaft of claim 6 each having a diameter of approximately 3 millimeters.

25. The cystoscopic needle holder and clip cutter of claim 1 having a camera located at the distal opening of one of the pair of channels.

26. The surgical device of claim 6 having a camera located at the distal opening of one of the pair of channels.
